(19) United States
(12) Patent Application Publication
(10) Pub. No.: US 2025/0114610 A1
(43) Pub. Date: Apr. 10, 2025

(54) SYSTEMS AND METHODS FOR MODULATING THE NEUROIMMUNE SYSTEM

(71) Applicant: Boston Scientific Neuromodulation Corporation, Valencia, CA (US)

(72) Inventors: Jianwen Gu, Richmond, TX (US); Bradley Lawrence Hershey, Carrollton, TX (US); Changfang Zhu, Valencia, CA (US)

(73) Assignee: Boston Scientific Neuromodulation Corporation, Valencia, CA (US)

(21) Appl. No.: 18/909,601

(22) Filed: Oct. 8, 2024

Related U.S. Application Data
(60) Provisional application No. 63/543,193, filed on Oct. 9, 2023.

(51) Int. Cl. A61N 1/36; A61N 1/372; A61N 5/06

(52) U.S. Cl. CPC A61N 1/36139; A61N 1/3606; A61N 1/37211; A61N 5/0622

(57) ABSTRACT

Methods and systems for modulating the neuroimmune response while monitoring biomarkers that may directly or indirectly correlate with neuroimmune function. A neuromodulation therapy may be delivered, and a biomarker level determined. Some biomarker levels are determined from bodily fluid collected from the patient, and are compared to a predetermined threshold level. A therapeutic stimulation is delivered to a patient using one or more electrode leads, and/or optical fibers.

### CROSS REFERENCE TO RELATED APPLICATIONS

The present application claims the benefit of U.S. Provisional Patent Application Ser. No. 63/543,193, filed Oct. 9, 2023, the disclosure of which is incorporated herein by reference.

### BACKGROUND

Numerous diseases involve neuroinflammation. Current medical treatment for neuroinflammation is primarily pharmacological, such as the use of anti-inflammatory drugs targeting cytokine-suppression, or microglial inhibition. Studies have shown that neuromodulation can modulate the neuroimmune system. Historically, neuromodulation therapies have not been optimized to reduce neuroinflammation. There is interest in optimizing neuromodulation therapy to target the neuroimmune response for reducing or otherwise treating neuroinflammation.

### OVERVIEW

The present inventors have recognized, among other things, that a problem to be solved is the need for non-pharmacological options for treating neuroinflammation. One solution is to optimize current neuromodulation devices for modulating neuroimmune system function. Modulating neuroimmune responses with electrical stimulation alone and/or in combination with light therapy is proposed, as well as monitoring neuroimmune biomarker levels. A closed loop system is also proposed, such as an SCS system that senses neuroinflammatory biomarkers in bodily fluid, or receives data regarding such biomarkers, and optimizes therapy over time. Some examples may include an implantable neuromodulation system used in combination with a sensing or other diagnostic system to obtain neuroinflammation markers such as by measuring biomarkers using interstitial fluid, blood, CSF or other bodily interaction.

A first illustrative and non-limiting example takes the form of a system for optimizing neural therapy for neuroimmune system function, the system comprising: an implantable pulse generator housing output circuitry configured to issue a neural therapy; a sensor for sensing a level one or more biomarkers in the patient; a controller, the controller configured to: instruct the output circuitry to issue a first neural therapy; instruct the sensor to measure the level of the one or more biomarkers following issuance of the first neural therapy by the implantable pulse generator; compare a measure of the post-neuromodulation level of the one or more biomarkers to a threshold; and instruct the output circuitry to issue a second neural therapy if the threshold is crossed by the measure.

Additionally or alternatively, the implantable pulse generator includes the sensor and the controller. Additionally or alternatively, the controller is housed in the implantable pulse generator and the sensor is separate from the implantable pulse generator, and communicates with the controller. Additionally or alternatively, the controller is in an external device having communication circuitry for communicating with the implantable pulse generator, optionally wherein the sensor is part of the external device.

Additionally or alternatively, the neural therapy comprises electrical pulses. Additionally or alternatively, the neural therapy comprises optical signals. Additionally or alternatively, the system further includes a lead coupled to the pulse generator and adapted to extend from the pulse generator to target neural tissue, wherein: the lead comprises one or more electrodes for outputting electrical pulses; and/or the lead comprises an optical transducer for generating an optical output from an electrical signal issued by the output circuitry; and/or the lead comprises an optical fiber for transmitting an optical signal from the output circuitry.

Additionally or alternatively, the measure is an amplitude of the biomarker, and the threshold is a population-based threshold for normal levels of the one or more biomarkers. Additionally or alternatively, the measure is an amplitude of the biomarker, and the threshold is a patient specific reference for the biomarker. Additionally or alternatively, the measure is a trend of the biomarker.

Additionally or alternatively, the sensor is configured to analyze a body fluid in-situ, and the body fluid is one of interstitial fluid, blood, cerebrospinal fluid, or intrathecal fluid.

Additionally or alternatively, the one or more biomarkers includes spleen tyrosine kinase. Additionally or alternatively, the one or more biomarkers includes Resolvin D1. Additionally or alternatively, the one or more biomarkers are selected from pro-inflammatory mediators, anti-inflammatory mediators, immune cells, or a Resolvin compound. Additionally or alternatively, the implantable pulse generator includes circuitry for measuring electrical signals from the patient's brain to determine an intrinsic gamma frequency of the patient, and the step of delivering a neural therapy to the patient comprises issuing electrical pulses at a repetition rate determined from the intrinsic gamma frequency of the patient.

Another illustrative, non-limiting example takes the form of a method of optimizing neural therapy for neuroimmune system function, the method comprising: delivering a neural therapy to a patient; determining a post-neuromodulation level of one or more biomarkers in the patient, wherein the level of the one or more biomarkers directly or indirectly correlates with neuroimmune system function; comparing a measure of the post-neuromodulation level of the one or more biomarkers to a threshold; finding that the threshold is crossed, and in response, adjusting a neural therapy setting.

Additionally or alternatively, the measure is an amplitude of the biomarker, and the threshold is a population-based threshold for normal levels of the one or more biomarkers. Additionally or alternatively, the measure is an amplitude of the biomarker, and the threshold is a patient specific reference for the biomarker. Additionally or alternatively, the measure is a trend of the biomarker.

Additionally or alternatively, the step of delivering a neural therapy to the patient is performed by issuing, from an implanted device, electrical stimuli to a neural, glial, and/or immune structures of the patient.

Additionally or alternatively, the step of determining a post-neuromodulation level of one or more biomarkers comprises collecting or analyzing bodily fluid in-situ. Additionally or alternatively, the bodily fluid is one of interstitial fluid, blood, cerebrospinal fluid, or intrathecal fluid.

Additionally or alternatively, one of the one or more biomarkers is spleen tyrosine kinase. Additionally or alternatively, the one or more biomarkers is Resolvin D1. Additionally or alternatively, the one or more biomarkers are selected from pro-inflammatory mediators, anti-inflammatory mediators, immune cells, or a Resolvin compound.

Additionally or alternatively, the step of delivering a neural therapy to the patient comprises issuing an optical modulation signal to neural tissue of the patient.

Additionally or alternatively, the method further includes measuring electrical signals from the patient's brain to determine an intrinsic gamma frequency of the patient, and the step of delivering a neural therapy to the patient comprises issuing electrical pulses at a repetition rate determined from the intrinsic gamma frequency of the patient.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a deep brain stimulation (DBS) system;

FIG. 2 shows a spinal cord stimulation (SCS) system; and

FIGS. 3A-3B show, in block form, a method for patient treatment.

### DETAILED DESCRIPTION

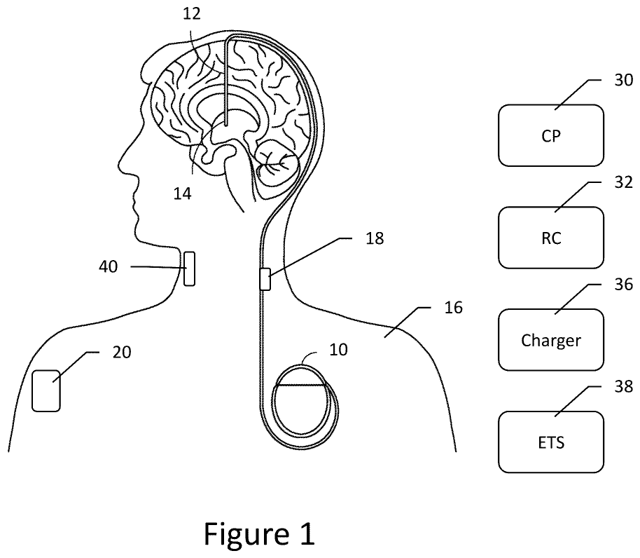

FIG. 1 shows an illustrative DBS system implanted in a patient. The system comprises an implantable pulse generator (IPG) 10, shown implanted in the pectoral region of a patient 16. The IPG 10 is coupled to a lead 12 which extends subcutaneously to the head of the patient 16, through a burr hole formed in the patient's skull, and then into the brain. In the example shown, the lead 12 includes a plurality of electrodes positioned near the distal end 14 of the lead. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, neuroinflammation, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

In addition to the preceding, some examples herein use a DBS system as illustrated in FIG. 1 to modulate neuroinflammation, such as by issuing therapy signals, or combinations of therapy signals, to affect neuroinflammation. Along with such treatment, biomarkers may be sensed, and the sensed biomarkers may be used to tailor or enhance therapy.

The illustrative system of FIG. 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. In other examples, a continuous wave energy may be delivered as, for example, a sinusoidal or DC or duty-cycled DC forms of continuous output. The CP 30 can be used by a physician, or at the direction of a physician, to obtain data from and provide instructions the IPG 10 via suitable communications protocols such as Bluetooth or MedRadio or other wireless communications standards, and/or via other modalities such as inductive telemetry.

A patient remote control (RC) 32 can be used by the patient to perform various actions relative to the IPG 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 32 can communicate via similar telemetry as the CP 30 to control and/or obtain data from the IPG 10. The patient RC 32 may also be programmable on its own, or may communicate or be linked with the CP 30.

A charger 36 may be provided to the patient to allow the patient to recharge the IPG 10, if the IPG 10 is rechargeable. Some IPG 10 are not rechargeable, and so the charger 36 may be omitted. The charger 36 can operate, for example, by generating a varying magnetic field to activate an inductor associated with the IPG 10 to provide power to recharge the IPG battery, using known methods and circuitry.

Some systems may include an external test stimulator (ETS) 38. The ETS 38 can be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (sometimes called an operating room cable) that couples to the ETS 38, and the ETS 38 can be programmed using the CP 30 with various therapy programs and stimulation parameters. Once therapy suitability for the patient is established to the satisfaction of the patient 16 and/or physician, the permanent IPG 10 is implanted and the lead 12 is connected thereto, with the ETS 38 then removed from use.

Additional features in the system of FIG. 1 may include a sensor 18 adapted to perform, for example, chemical testing. For example, an optical sensor may include a light source and cooperating light sensor, each using wavelength selective interrogation to determine chemical constituents of surrounding fluid. In another example, a chemical sensor 18 can be provided, as desired. The sensor 18 may be tethered the IPG 10 on a separate element, or may reside on one of the leads 12. The sensor 18 may instead be provided separate from the IPG 10, and may communicate such as by conducted communication, sonic or ultrasonic signaling, or wireless communication such as Bluetooth, Medradio or other RF, as desired. A separately provided sensor 18 may operate responsive to an interrogation or activation signal, such as by receiving power inductively or via an RF or ultrasound signal, if desired. The sensor 18, as described, may provide in-situ testing of a bodily fluid to determine levels of one or more biomarkers as described below. Rather than a separately housed sensor 18, the IPG 10 may include the circuitry and interrogation or gathering apparatuses, such as a chemical sensor or optical interface for measuring and/or determining levels of a biomarker. A biomarker “amplitude” may indicate the prevalence, such as concentration, whether relative or absolute, in bodily fluid and any suitable parameterization of a biomarker may be used.

A vagal stimulation system may be provided as shown at 40, located near the vagus nerve. This may be in place of the IPG 10 and lead 12, if desired, or may be an additional stimulator for the patient 16. Stimulation devices may be microstimulators, and may include or omit a lead, as desired. Some example microstimulators are disclosed in U.S. Pat. No. 8,127,424, the disclosure of which is incorporated herein by reference. Devices, including microstimulators, may be externally powered or internally powered, as desired.

An external sensing system may be provided as indicated at 20. For example, an external sensor may be used to obtain a fluid sample, such as interstitial fluid, blood, lymph, cerebrospinal fluid (CSF), etc. that can be analyzed to identify chemical constituents. The results may then be used to adjust, trigger, or modulate/pause/delay therapy using the implanted systems 10/12 or 40, as desired.

In some examples, the internal sensor 18 or external sensing system 20 may be a fluid analyzer for determining concentration or other measurable levels of biomarkers in fluid obtained from a patient, such as by blood or other fluid draw, saliva, urine, etc. Additionally or alternatively, either sensor 18, 20 may take the form of an optical analyzer or combination optical interrogating and analysis system. For example, an optical interrogating and analysis system may output one or several wavelengths of light using, for example, light emitting diodes, lasers, vertical cavity surface emitting lasers, etc., and the observing light refraction, reflection, or pass-through of patient fluid or tissue. The wavelengths used can determine what analytes of tissue or fluid are measured, for example. Any sensor capable of determining the concentration and/or trends of the biomarkers identified herein can be used as internal sensor 18 or external sensing system 20. It should be noted that some systems only include one of the two sensors 18, 20, though it is also possible to include or use both, for example, for ongoing monitoring and/or to periodically calibrate or check accuracy of an implanted sensor 18 using an external sensor 20. Some examples refer to the “amplitude” of a biomarker; this should be understood as referring broadly to a measurable quantity of the biomarker, such as a concentration or prevalence, or simple presence, in the tissue or bodily fluid being analyzed.

The external sensing system 20 or, alternatively, a sensor 18 that is implanted in the patient, may communicate with an external controller, such as the CP 30 or RC 32, which in turn issues command signals to the IPG 10, to adjust therapy or issue a second therapy after a first therapy, where “adjust” may include any of changing a therapy parameter, activating therapy, or causing therapy to cease. Thus, there is a controller in the external device that communicates with both a sensor (18 or 20) and the IPG 10, where the IPG includes output circuitry for issuing therapy to the patient (such as electrical or optical therapy, or both, as described herein). In another example, the sensor 20 may be part of a CP 30 or RC 32, if desired. Alternatively, an IPG may integrate the sensor 18 and also include a controller that performs analysis to determine whether and when to issue further therapy or adjust therapy in response to sensed biomarker data. In still other examples, the IPG 10 may have a controller and output circuitry for delivery neural therapy to the patient, and the IPG includes communication circuitry for communicating with a sensor (implanted 18 or external 20) to receive biomarker data.

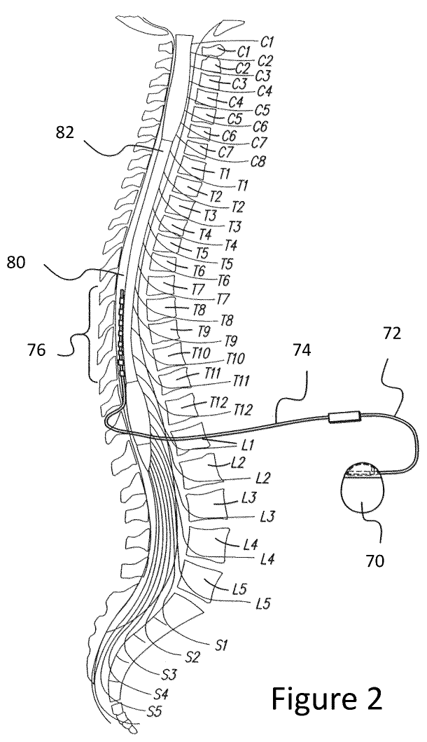

FIG. 2 shows an illustrative spinal cord stimulation system as implanted. In this example, an IPG 70 may be placed near the buttocks or in the abdomen of the patient, with or without a lead extension 72 for coupling to the lead(s) 74 that enter the spinal column. Region 76 at about the level of the lower thoracic or upper lumbar vertebrae may serve as an entry point to the spinal column, where the distal end of the lead 74 with an electrode array may be placed close to the spinal cord 80. Other locations for the IPG 70 and/or lead 74 may be used. For example, sacral nerve stimulation may be performed.

The standard approach to therapy in systems similar to those shown in FIGS. 1-2 has been that the IPG 14 (and ETS 20) may deliver current controlled or voltage-controlled therapy comprising either biphasic square waves or monophasic square waves having passive recovery. In general, the amount of current out of an electrode should zero out over time to avoid encouraging corrosion at the electrode-tissue interface. For this reason, biphasic pulses, or monophasic pulses with a passive recovery period are typically used.

Much of the therapy delivered in DBS and/or SCS is provided with no feedback or only indirect feedback. Much of the feedback is also instantaneous and acute, which will not provide visibility to chronic or longer-term changes. For example, during some testing and configuration procedures, a patient may consciously respond to questions regarding therapy benefits or side effects, interposing both the delay time needed for a patient to perceive stimulus effects, as well as adding uncertainty due to subjective factors to the feedback. Sensing and modulating neuroinflammation information is highly desired, and some examples herein use additional testing and/or sensing to achieve a closed loop neuroinflammation therapy.

Numerous diseases involve neuroinflammation (e.g. chronic pain, Parkinson's disease, Alzheimer's disease, epilepsy, major depression, type 2 diabetes, rheumatoid arthritis, hypertension, irritable bowel syndrome, asthma etc.). It would be advantageous to optimize existing neuromodulation devices to modulate the neuroimmune response for anti-neuroinflammation. For example, some neuromodulation systems (DBS, SCS, Vagus Nerve, etc.) have been shown to modulate the neuroimmune system.

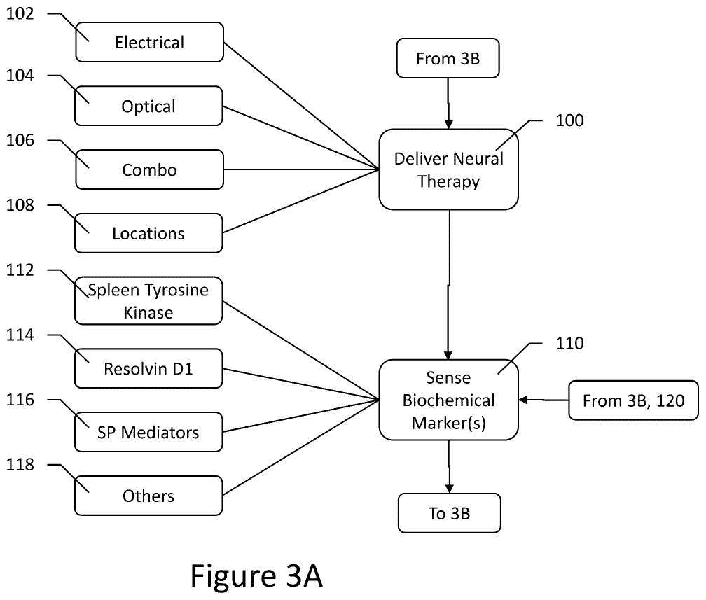

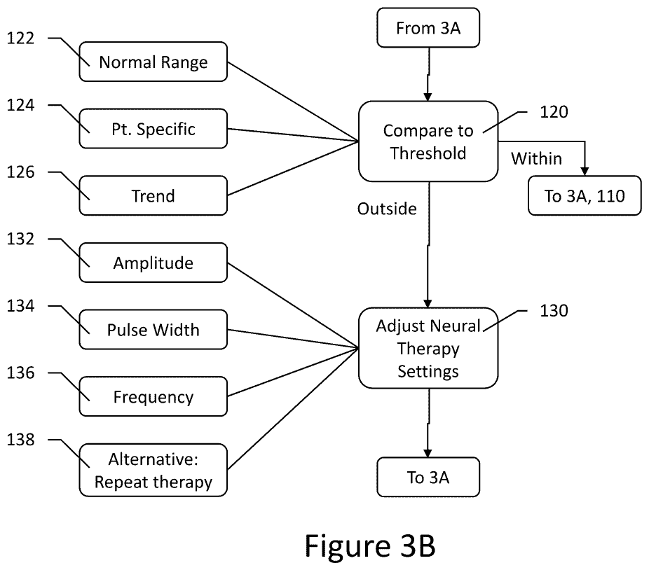

FIGS. 3A-3B show in block form, an illustrative process of delivering neural therapy, sensing biochemical markers, comparing to threshold, and adjusting a neural therapy setting. Starting in FIG. 3A, at block 100, neural therapy is delivered by a neuromodulation system as described above. Therapy may be delivered as solely electrical therapy 102, as optical therapy 104, or as a combination 106 of electrical and optical therapy, or combined with still other therapy as desired (magnetic, ultrasonic, etc.).

The therapy location in block 108 may be to any one of the previously mentioned “Targets.” For example, therapy may be directed to brain structures such as the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Stimulation may instead be directed to the spinal cord, occipital nerve, Vagus nerve, a renal nerve, sacral nerve, or other location. In some embodiments, the therapy may be delivered to more than one location 108.

Electrical stimulation 102, optical stimulation 104, and/or combined stimulation may be defined by any suitable parameters or combinations of parameters, and varied/control by amplitude of stimulation pulses, frequency or repetition rate of stimulation pulses, pulse width of stimulation pulses, and more complex parameters such as burst definition. Biphasic square waves may be used, and/or ramped, triangular, sinusoidal, multiphasic, monophasic and other stimulation pulses or types may be used as desired, including continuous wave energy which may be delivered as, for example, a sinusoidal or DC or duty-cycled DC forms of continuous output, which can be used to reduce neuroinflammation when applied to any suitable neural structure. Optical therapy 104, alone or as a combination 106, may include any variation of neuromodulation device, such as using an optical source or transducer(s) at the end of a lead, or using a lead having an optical fiber passing from a pulse generator in which a light source(s) is located. In some embodiments a light source may be located on a lead extension body. In some embodiments, the optical source may be configured to dose light with a wavelength in the range of about 600 nanometers (nm) to 1300 nm to a target, though longer or shorter wavelengths may also be used, as desired.

Stimulation for purposes of neuroinflammation therapy may occur using different schedules than are used with some other systems. For example, rather than issuing therapy for hours each day, a neuroinflammation therapy may be delivered at a duty cycle of, for example, 1% or less. For example, stimulation can be delivered for about 1 to about 10 minutes per day, each day, or may be delivered less frequently than daily, in some examples. In another example, stimulation may last for several hours, but only occur once every seven to thirty days. Other variations of low duty cycle stimulation may be used; the “duty cycle” indicates the quantity of time that stimulation is on relative to the overall repetition rate for a given therapy program.

At block 110, biochemical markers for neuroinflammation are sensed. Biomarkers may include spleen tyrosine kinase (STK) 112, resolvin D1 (RvD1) 114, special preresolving mediators (SP mediators) 116, such as SP mediators derived from omega 3 polyunsaturated fatty acids, or other biomarkers 118 as desired. Other biomarkers 118 may include, for example, interleukin1 beta, and/or the neutrophil-derived chemical marker S100-8B. Other biomarkers 118 may further include pro-inflammatory and anti-inflammatory mediators such as chemokines and cytokines. Other biomarkers 118 may also include immune cells such as monocytes, macrophages, T-cells, phagocytes, and lymphocytes among others. In addition to RvD1, other forms of Resolvin may be used as biomarkers such as Resolvin E1 Resolvin E3, Resolvin E4, 17R-Resolvin D1, Resolvin D2, Resolvin D3, 17R-Resolvin D3, Resolvin D4, and Resolvin D5. Some biomarkers may be associated with the innate immune system, if desired.

These and other biomarkers may be used to determine a level of neuroinflammation and oxidative stress as described in more detail below. In some embodiments one or more biomarker levels may be measured by a wearable device. In some embodiments one or more biomarker levels may be measured from a blood draw. In some embodiments, one or more biomarker levels may be measured intrathecally. In some embodiments the level of biomarker may be inferred from sympathetic or parasympathetic tone (blood pressure, heart rate etc.). For example, increased sympathetic responses (high heart rate and/or blood pressure) may relate to increased neuroinflammation. In some embodiments, biomarkers may be assessed using tomography to assess local blood flow and/or oxygenation. In some embodiments, one or more biomarker levels may be measured by a wearable device that issues stimuli that cause electroporation and release or increase of the presence of interstitial fluid, and the interstitial fluid may be obtained for chemical analysis, such as via a biomarker assay. In some embodiments the biomarker levels may be tested daily. In other embodiments the biomarker levels may be tested more infrequently, such as every other day, once a week, etc. Biomarkers may be tested in response to an event identified by the patient or by a device; for example, if a patient experiences a period of increased anxiety, the patient may respond by obtaining a biomarker sample using a wearable device. In some embodiments the biomarker levels may be tested frequently at the onset of a new therapy setting, and then decrease in frequency as time passes.

Turning to FIG. 3B, at block 120, the measured levels of neuroinflammation biomarker are compared to a predetermined threshold. Such thresholds may be external and/or absolute, such as using a population-based definition of a “normal reference” or “normal range,” as indicated at 122. In other examples the threshold may be patient specific, as indicated at 124. For example, a patient-specific threshold may be determined by observing patient symptoms linked to neuroinflammatory states, and identifying levels of a given biomarker at which symptoms onset or subside, and using the identified levels as a threshold or target. Patient-specific threshold or reference values may include statistical measures, for example, an average of a particular marker over time, plus or minus one or more of variance, standard deviation, etc. A trend 126, such as may be determined from a rate of change of the level of neuroinflammation biomarker may also be assessed, as well as the direction of change of the neuroinflammation biomarker. For example, it may be determined if the level of biomarker is increasing at a high rate. Combinations of such analyses may be used, such as observing whether a biomarker is outside of a population based normal range 122, or outside of a patient specific expected range 124 with a trend 126 toward the outer boundary of the normal range 122, either of which may be deemed a trigger in an example. Trend 126 may also look for an absolute change in a specific period of time of any biomarker of interest.

If the analysis at 120 crosses or is outside whichever threshold (population-based normal 122, patient specific 124, and/or trend 126) is applicable, therapy parameters may be adjusted, as indicated at 130. At block 130, therapy parameters may be adjusted according to biomarker information. Adjustments may include changes to amplitude 132, pulse width 134, duty cycle (not shown), and frequency 136. Stimulation may be started or stopped, or repeated, as another type of adjustment at 130. For example, combination stimulation at 60 Hz and 1 kHz with arbitration-induced pulse patterning may reduce neuroinflammation by increasing the production and release of RvD1. In another example, stimulation at 40 Hz may increase levels of SYK and decrease neuroinflammation.

Therapy may be delivered at the patient's intrinsic gamma frequency to decrease neuroinflammation, for example, by first obtaining, from an implanted device such as a lead, or by other invasive data collection (such as a sensing catheter advanced into a blood vessel in the brain), or by transcranial sensing, electrical signals emanating from the brain. The patient's gamma frequency can then be determined by analysis, such as a Fourier transformation, principal components analysis, wavelet decomposition, and other methods that can identify frequencies of interest in the captured signals. Therapy can be issued with, for example, a pulse repetition rate, or continuous wave energy such as a sinusoidal or other continuous output, or any other waveform configured to match the patient's determined gamma frequency.

In yet another example, low level light irradiation in red to near-infrared (600 nm-1300 nm) has been shown to have an anti-inflammatory effect on the brain. In any such example, one or more of frequency, duty cycle, duration of therapy, amplitude, pulse width, targeted location of therapy, etc. may be adjusted. The therapy cycle may be considered a chronic therapy, so that continuing updates and adjustments over time are likely.

In some examples, rather than using the procedure of FIGS. 3A-3B to adjust therapy, the approach may be to trigger additional therapy. For example, if the biomarker resides within the threshold, block 120 may return the process to block 110, for additional sensing of the biochemical marker(s), such as after waiting some period of time (a day, or several days, for example). As the biomarker is monitored, a trend 126 in biomarker data may be observed, and used as an additional way of determining whether the threshold is crossed at block 120. Multiple thresholds may apply, for example, a first threshold may relate to determining whether therapy caused an effect when delivered, and a second to determine when repeat therapy is needed after successful initial treatment. When the second threshold is crossed at block 120, meaning the patient is due for additional therapy, block 130 may include repeating the previously delivered therapy, as indicated at 138. This can be achieved by preserving existing therapy parameters and returning to block 110 from FIG. 3B/block 130, and re-issuing the therapy.

Throughout the process, biomarker levels may also be stored, and changes in biomarker level after neural therapy changes may affect subsequent therapies. For example, if a neural therapy is adjusted to increase RvD1 levels and RvD1 levels increase slowly, the therapy parameters may be adjusted to increase the rate of change. In another example, if red to near-infrared light is dosed to a patient, and neuroinflammatory biomarker levels don't change, the therapy parameters may be adjusted to include another type of treatment e.g. electrical stimulation. Such linked data may be reported to a central repository or database to inform therapy decisions for other or future patients, if desired.

In some embodiments, a neuromodulation therapy regimen may be adjusted to include the above described stimulation parameters at a certain interval. In some embodiments, the neural therapy may only be implemented for a few hours. In other embodiments, the neural therapy may be implemented at a certain interval (weekly, monthly etc.). In another embodiment, the therapy may be dosed for a few hours on days 1, 3, and 5 of a treatment cycle. In any of the above described embodiments, the therapy may be followed by biomarker monitoring, which may trigger further therapeutic treatments or adjustments to treatment parameters.

For example, a biomarker of neuroinflammation may be measured prior to treatment to determine a baseline for the patient's condition. Therapy may be issued to modulate the neuroinflammatory system, and the biomarker may be monitored following therapy to observe a change therein, where the change may be a decrease in a neuroinflammatory agent, or an increase in a neuroinflammatory inhibitor. As the observed change begins to shrink, and the patient returns to the baseline, subsequent therapy can be triggered. Illustrative timeframes may vary depending on the therapy used and the underlying marker being monitored, and may range from minutes, to hours, days, or weeks, or longer.

For example, if a normalized baseline metric is set at 10 for a given neuroinflammatory agent for a particular patient, therapy may reduce the metric to a normalized score of, for example, 1. After therapy, over time, the metric may return toward the normalized baseline. When the metric reaches a predetermined threshold, such as 3, 5, 7, 9, depending on physician choices and/or perceived effect of the agent that the metric represents (such as onset of symptoms), therapy can be again triggered.

Conversely, if a normalized baseline metric is set to 0 for a given neuroinflammatory inhibitor, therapy may increase the metric to, for example, 10. After therapy, over time, the metric may return toward the normalized baseline. When the metric reaches a predetermined threshold, such as 6, 4, 3, 2, depending on physician choices and/or perceived effect of the inhibitor that the metric represents (such as the onset of symptoms), therapy can again be triggered.

Some examples may use therapies having effects that can be relatively long lasting. For example, a combined therapy of relatively lower frequency stimulation issued to one tissue area, and relatively higher frequency stimulation issued to a second tissue area, may have long lasting effects. The lower frequency may be in the range of up to 100 Hz, or in the range of 40-60 Hz, for example, lasting for minutes to hours. The higher frequency may exceed 200 Hz, or 500 Hz, or 1000 Hz, for example, in the range of 1 kHz to 2 kHz, or about 1.2 kHz, issued for minutes to hours as well. Such a combination has been shown to provide modulatory effects for up to several weeks. A specific combination may be to offer overlapping therapy with a 40-60 Hz lower frequency signal, and a 1.2 kHz higher frequency delivered to neural tissue. The two therapies may be directed to a single tissue location or to separate tissue locations, as desired, such as in a single or more than one location along the spinal cord.

A communication circuit may include for example, an application specific integrated circuit (ASIC), such as commercially available Bluetooth and/or Bluetooth Low Energy chips, or a Medradio ASIC (also commercially available), or a set of discrete components including at least an oscillator, antenna, and tuning circuitry adapted to generate an RF or other frequency level signal. Inductive telemetry, using an inductive coil, or other telemetry circuitry may be used instead, including conducted communication and/or any of optical or mechanical (ultrasound, for example) communication. A controller may include a microcontroller, microprocessor, or state machine logic, associated with logic and/or memory for storing instructions in machine readable format in tangible and/or non-transient format (such as Flash, RAM, ROM, etc.) to perform methods as described herein. Output circuitry may include, for example, current controller or voltage-controlled output circuitry known in various commercially available SCS, DBS and/or Vagus Nerve stimulation systems.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of optimizing neural therapy for neuroimmune system function, the method comprising:
delivering a neural therapy to a patient;
determining a post-neuromodulation level of one or more biomarkers in the patient, wherein the level of the one or more biomarkers directly or indirectly correlates with neuroimmune system function;
comparing a measure of the post-neuromodulation level of the one or more biomarkers to a threshold;
finding that the threshold is crossed, and in response, adjusting a neural therapy setting.

2. The method of claim 1, wherein the measure is an amplitude of the biomarker, and the threshold is a population-based threshold for normal levels of the one or more biomarkers.

3. The method of claim 1, wherein the measure is an amplitude of the biomarker, and the threshold is a patient specific reference for the biomarker.

4. The method of claim 1, wherein the measure is a trend of the biomarker.

5. The method of claim 1, wherein the step of delivering a neural therapy to the patient is performed by issuing, from an implanted device, electrical stimuli to a neural, glial, and/or immune structures of the patient.

6. The method of claim 1, wherein the step of determining a post-neuromodulation level of one or more biomarkers comprises collecting or analyzing bodily fluid in-situ.

7. The method of claim 6, wherein the bodily fluid is one of interstitial fluid, blood, cerebrospinal fluid, or intrathecal fluid.

8. The method of claim 1, wherein one of the one or more biomarkers is spleen tyrosine kinase.

9. The method of claim 1, wherein the one or more biomarkers is Resolvin D1.

10. The method of claim 1, wherein the one or more biomarkers are selected from pro-inflammatory mediators, anti-inflammatory mediators, immune cells, or a Resolvin compound.

11. The method of claim 1, wherein the step of delivering a neural therapy to the patient comprises issuing an optical modulation signal to neural tissue of the patient.

12. The method of claim 1, further comprising measuring electrical signals from the patient's brain to determine an intrinsic gamma frequency of the patient, and the step of delivering a neural therapy to the patient comprises issuing electrical pulses at a repetition rate determined from the intrinsic gamma frequency of the patient.

13. A system for optimizing neural therapy for neuroimmune system function, the system comprising:
an implantable pulse generator housing output circuitry configured to issue a neural therapy;
a sensor for sensing a level one or more biomarkers in the patient;
a controller, the controller configured to:
instruct the output circuitry to issue a first neural therapy;
instruct the sensor to measure the level of the one or more biomarkers following issuance of the first neural therapy by the implantable pulse generator;
compare a measure of the post-neuromodulation level of the one or more biomarkers to a threshold; and
instruct the output circuitry to issue a second neural therapy if the threshold is crossed by the measure.

14. The system of claim 13, wherein the implantable pulse generator includes the sensor and the controller.

15. The system of claim 13, wherein the controller is housed in the implantable pulse generator and the sensor is separate from the implantable pulse generator, and communicates with the controller.

16. The system of claim 13, wherein the controller is in an external device having communication circuitry for communicating with the implantable pulse generator.

17. The system of claim 16, wherein the sensor is part of the external device.

18. The system of claim 13, wherein the neural therapy comprises electrical pulses.

19. The system as claim 18, further comprising a lead coupled to the pulse generator and adapted to extend from the pulse generator to target neural tissue, wherein the lead comprises one or more electrodes for outputting electrical pulses.

20. The system of claim 13, wherein the neural therapy comprises optical signals.
